(19) United States
(12) Patent Application Publication
(10) Pub. No.: US 2008/0317697 A1
(43) Pub. Date: Dec. 25, 2008

(54) Shaving kit and method of shaving comprising a personal care composition and an in shower body lotion

(76) Inventors: Thomas Arthur Sturgis, Mason, OH (US); Jeannic Tung, Mason, OH (US); Kim Michaelle McMillan, Mason, OH (US); Bhavna Ranjit Chatterjee, Indian Hill, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 11/820,817

(22) Filed: Jun. 21, 2007

(51) Int. Cl. A61K 8/00; A61Q 9/00

(52) U.S. Cl. 424/73

(57) ABSTRACT

The present invention relates to a shaving kit that comprises a first personal care article, a second personal care article and a shaving razor. The first personal care article comprises a personal care composition contained within a package. The personal care composition comprises a surfactant, water and at least 15% of a hydrophobic moisturizing material. The second personal care article comprises an in-shower body lotion composition contained in a package; and a set of instructions in association with the package. The set of instructions comprising instructions to dispense the in-shower body lotion composition from the package, contact the skin surface with the in-shower body lotion composition during showering or bathing, rinse the skin surface, and dry the skin surface. The present invention relates to a method of shaving and a personal care article.

### FIELD OF THE INVENTION

The present invention relates to a shaving kit, a method of shaving and a personal care article.

### BACKGROUND OF THE INVENTION

Consumers who shave typically use aerosol foaming compositions or gels. These foaming compositions provide a cushion and lubricate the skin during shaving. Unfortunately, some aerosol foaming compositions and gels have a tendency to dry the skin. Non-aerosol foaming compositions can be used to lather and lubricate the skin for wet shaving, yet may not provide a satisfactory degree of moisturization. Hydrophobic moisturizing materials, like petroleum, are well known skin lubricants and moisturizers. However, adding hydrophobic moisturizing materials to surfactant based non-aerosol compositions causes the compositions to lather poorly when hydrophobic moisturizing materials are introduced into the composition at levels sufficient to lubricate the skin for shaving. Moreover, compositions that largely comprise hydrophobic moisturizing material tend to clog the shaving razor causing poor performance. Therefore, even though aerosol compositions and gels can dry their skin, consumers have continued to use these compositions out of habit and to avoid clogging their razor and blades.

While moisturizing personal care compositions which include hydrophobic moisturizing materials show improved skin conditioning benefits during shaving over gels, aerosols, and other shaving products, the full potential benefits of personal care compositions is not achieved because most of the personal care composition is being removed by the razor during shaving. In fact, it is believed that some of the stratum corneum is removed with the product which reduces the moisture barrier of the skin. Thus, it is important for consumers to use a personal care composition to lock in moisture in a short period of time after shaving. Accordingly, the need still remains for a non-aerosol personal care composition that lathers well and lubricates the skin while providing improved skin conditioning benefits for use during shaving used in concert with another personal care composition with hydrophobic moisturizing material after shaving.

### SUMMARY OF THE INVENTION

The inventors believe that the present invention provides a kit that comprises a personal care composition that lathers well, and lubricates in combination with an in-shower body lotion composition, razor blade cartridges, a shaving razor or instructions for shave, which illustrate to consumers that the compositions are suitable for use in shaving, as well as, suitable for use in cleansing, as a body wash. The use of the in-shower body lotion composition is designed to lock in the moisture from the shower and increase skin conditioning benefits, improving the moisture barrier properties of skin in spite of the act of shaving.

The present invention relates to a shaving kit that comprises a first personal care article, a second personal care article and a shaving razor. The first personal care article comprises a personal care composition contained within a package. The personal care composition comprises a surfactant, water and at least 15% of a hydrophobic moisturizing material. The second personal care article comprises an in-shower body lotion composition contained in a package; and a set of instructions in association with the package. The set of instructions comprises instructions to dispense the in-shower body lotion composition from the package, contact the skin surface with the in-shower body lotion composition during showering or bathing, rinse the skin surface, and dry the skin surface. The present invention also relates to a method of shaving and a personal care article.

### BRIEF DESCRIPTION OF THE DRAWINGS

FIG. 1 is a graph that plots the change of dryness of skin, as graded by an expert grader, three hours after using various shaving regimes.

FIG. 2 is a graph that plots the change of dryness of skin, as graded by an expert grader, three hours after using three shaving regimes including the combination of a personal care composition and a razor, the kit of the present invention and no treatment.

FIG. 3 is a graph that plots the change of dryness of skin, as graded by an expert grader, twenty-four hours after using various shaving regimes.

FIG. 4 is a graph that plots the change of dryness of skin, as graded by an expert grader, twenty-four hours after using three shaving regimes including the combination of a personal care composition and a razor, the kit of the present invention and no treatment.

FIG. 5 is a graph that plots the change in transepidermal water loss of the skin three hours after various shaving regimes.

FIG. 6 is a graph that plots the change in transepidermal water loss of the skin three hours after three shaving regimes including the combination of a personal care composition and a razor, the kit of the present invention and no treatment.

FIG. 7 is a graph that plots the change in transepidermal water loss of the skin twenty-four hours after various shaving regimes.

FIG. 8 is a graph that plots the change in transepidermal water loss of the skin twenty-four hours after three shaving regimes including the combination of a personal care composition and a razor, the kit of the present invention and no treatment.

### DETAILED DESCRIPTION OF THE INVENTION

### I. Definitions

The term “anhydrous” as used herein, unless otherwise specified, refers to those compositions or materials containing less than about 10%, more preferably less than about 5%, even more preferably less than about 3%, even more preferably zero percent, by weight of water.

The term “cleansing implement” as used herein is meant as the device, or instrument used in performing the task of cleansing. Suitable cleansing implements include but are not limited to cleansing puff, sponge, brush, wash cloth, disposable cloth, and the like.

The term “multiphase” as used herein means that compositions comprise at least two phases which are chemically distinct (e.g. a surfactant phase and a benefit phase). These phases are in direct physical contact with one another and are not separated by a barrier. In one aspect of the invention, phases of the multiphase personal care composition are blended or mixed to a significant degree. In another aspect of the invention, the phases of the multiphase personal care composition are made to occupy separate but distinct physical spaces inside the package in which they are stored, but are in direct contact with one another (i.e., they are not separated by a barrier and they are not emulsified or mixed to any significant degree). In one preferred embodiment of the present invention, the “multi-phase” personal care compositions comprise at least two visually distinct phases which are present within the package as a visually distinct pattern. The pattern results from the combination of the “multi-phase” composition by a method of manufacture herein described. The “patterns” or “patterned” include but are not limited to the following examples: striped, marbled, rectilinear, interrupted striped, check, mottled, veined, clustered, speckled, geometric, spotted, ribbons, helical, swirl, arrayed, variegated, textured, grooved, ridged, waved, sinusoidal, spiral, twisted, curved, cycle, streaks, striated, contoured, anisotropic, laced, weave or woven, basket weave, spotted, and tessellated. The ratio of a first phase to a second phase can be from about 90:10 to about 10:90, from about 80:20 to about 20:80, from about 70:30 to about 30:70, from about 60:40 to about 40:60, and about 50:50.

The term “package” includes any suitable container for personal care compositions exhibiting a viscosity from about 1,500 centipoise (cP) to about 1,000,000 cP, including but not limited to a bottle, tottle, tube, jar, non-aerosol pump and mixtures thereof. As used herein “tottle” refers to a bottle which rests on the neck or mouth which its contents are filled in and dispensed from, but it is also the end upon which the bottle is intended to rest or sit upon for storage by the consumer and/or for display on the store shelf, as described in the commonly owned U.S. patent application Ser. No. 11/067443 filed on Feb. 25, 2005 to McCall, et al, entitled “Multi-phase Personal Care Compositions, Process for Making and Providing, and Article of Commerce.”

The term “personal care composition” as used herein, refers to compositions intended for topical application to the skin or hair. The compositions of the present invention are rinse-off formulations, in which the product is applied topically to the skin or hair and then is subsequently rinsed within minutes from the skin or hair with water, or otherwise wiped off using a substrate with deposition of a portion of the composition. The compositions also may be used as shaving aids. The personal care composition of the present invention is typically extrudable or dispensible from a package. The personal care compositions typically exhibit a viscosity of from about 1,500 centipoise (cP) to about 1,000,000 cP, as measured by as measured by the Viscosity Method as described in the commonly owned, patent application published on Nov. 11, 2004 under U.S. Publication No. 2004/0223991A1 by Wei, et al. The personal care compositions of the present invention can be in the form of liquid, semi-liquid, cream, lotion or gel compositions intended for topical application to skin. Examples of personal care compositions of the present invention can include but are not limited to shampoo, conditioning shampoo, body wash, moisturizing body wash, shower gels, skin cleansers, cleansing milks, hair and body wash, pet shampoo, shaving preparations and cleansing compositions used in conjunction with a disposable cleansing cloth.

A “skin compatible oil”, as defined herein, is an oil that is liquid or semi-solid at the temperature at which bathing is carried out that is deemed safe for use in cosmetics being either inert to the skin or actually beneficial. The most useful skin compatible oils for the present invention include ester oils, hydrocarbon oils, and silicone oils. Examples of these skin compatible oils are described in U.S. Publication No. 2004/0223992, U.S Publication No. 2003/0190296, U.S Publication No. 2003/0054019, U.S Publication No. 2003/0049282, U.S. Pat. No. 6,699,488 and U.S. Pat. No. 6,645,511.

The term “stable,” as used herein, means that the personal care composition comprises less than 5% “third-phase” volume, more preferably less than 2% “third-phase” volume, most preferably less than 1% “third-phase” volume after undergoing the rapid protocol aging and third phase measurement as described in the “Third-Phase” Method disclosed in co-pending U.S. application Ser. No. 11/312615 entitled “Shaving Kit, Article of Commerce and Method of Shaving Comprising a Personal Care Composition” filed Dec. 20, 2005.

The term “structured,” as used herein means having a rheology that confers stability on the composition. The degree of structure is determined by characteristics determined by one or more of the following methods the Young's Modulus Method, Yield Stress Method, or the Zero Shear Viscosity Method or by the Ultracentrifugation Method, all disclosed in co-pending U.S. application Ser. No. 11/312615 entitled “Shaving Kit, Article of Commerce and Method of Shaving Comprising a Personal Care Composition” filed Dec. 20, 2005. Accordingly, a surfactant phase of the personal care composition of the present invention is considered “structured,” if the surfactant phase has one or more of the following properties described below according to the Young's Modulus Method, Yield Stress Method, or the Zero Shear Viscosity Method or by the Ultracentrifugation Method. A surfactant phase is considered to be structured, if the phase has one or more of the following characteristics:
A. a Yield Stress of greater than about 0.1 Pascal (Pa), more preferably greater than about 0.5 Pa, even more preferably greater than about 1.0 Pa, still more preferably greater than about 2.0 Pa, still even more preferably greater than about 3 Pa, and even still even more preferably greater than about 5 Pa as measured by the Yield Stress and Zero Shear Viscosity Method described hereafter:
B. a Zero Shear Viscosity of at least about 500 Pascal-seconds (Pa-s), preferably at least about 1,000 Pa·s, more preferably at least about 1,500 Pa·s, even more preferably at least about 2,000 Pa·s; or
C. a Structured Domain Volume Ratio as measured by the Ultracentrifugation Method described hereafter, of greater than about 40%, preferably at least about 45%, more preferably at least about 50%, more preferably at least about 55%, more preferably at least about 60%, more preferably at least about 65%, more preferably at least about 70%, more preferably at least about 75%, more preferably at least about 80%, even more preferably at least about 85%; or
D. A Young's Modulus of greater than about 10 Pascal (Pa), more preferably greater than about 50 Pa, even more preferably greater than about 75 Pa, and still more preferably greater than 100 Pa.

The phrase “substantially free of” as used herein, unless otherwise specified means that the composition comprises less than about 5%, less than about 3%, less than about 1% and less than about 0.1% of the stated ingredient. The term “free of” as used herein means that the composition comprises 0% of the stated ingredient which has not been added to the composition, however, these ingredients may incidentally form as a byproduct or a reaction product of the other components of the composition.

The term “surfactant component” as used herein means the total of all anionic, nonionic, amphoteric, zwitterionic and cationic surfactants in a phase. When calculations are based on the surfactant component, water and electrolyte are excluded from the calculations involving the surfactant component, since surfactants as manufactured typically are diluted and neutralized.

The term “visually distinct” as used herein, refers to a region of the personal care composition having one average composition, as distinct from another region having a different average composition, wherein the regions are visible to the unaided naked eye. This would not preclude the distinct regions from comprising two similar phases where one phase could comprise pigments, dyes, particles, glitter, pearlescence and various optional ingredients, hence a region of a different average composition. A phase generally occupies a space or spaces having dimensions larger than the colloidal or sub-colloidal components it comprises. A phase can also be constituted or re-constituted, collected, or separated into a bulk phase in order to observe its properties, e.g., by centrifugation, filtration or the like.

### II. Shaving Kit

The present invention relates to a shaving kit that comprises a first personal care article, a second personal care article and a shaving razor. The first personal care article comprises a personal care composition contained within a package. The personal care composition comprises a surfactant, water and at least 15% of a hydrophobic moisturizing material. The second personal care article comprises an in-shower body lotion composition contained in a package; and a set of instructions in association with the package. The set of instructions comprises instructions to dispense the in-shower body lotion composition from the package, contact the skin surface with the in-shower body lotion composition during showering or bathing, rinse the skin surface, and dry the skin surface.

The shaving kit can further comprise a storage device for storing the shaving razor and plurality of razor blade cartridges. Suitable storage devices are those such as those disclosed in U.S. Pat. No. 6,415,517 issued to Charles Brigham Worrick, III on Jul. 9, 2002. The shaving kit can comprises a dispenser for dispensing a plurality of razor blade cartridges, such as those disclosed in U.S. Pat. No. 3,783,493 issued to Dawidowicz, et al on Jan. 8, 1974. The shaving razor, handle of the shaving razor, the razor blade cartridges, storage device, or dispenser may match in color the personal care composition or one of the phases of the personal care composition.

The shaving kit of can comprise a set of instructions comprising the method of shaving described below. The shaving kit can comprise one or more additional packages containing a personal care composition selected from the group consisting of a shampoo, conditioner, body lotion, skin care composition, deodorant, antiperspirant, after shave lotion and mixtures thereof. The additional packages can be a size selected from the group consisting of full size, travel size, trial size and mixtures thereof. The shaving kit, first personal care article, and second personal care article can further comprise a coupon, rebate, or advertisement. The coupon, rebate or advertisement is associated with one or more of the personal care products selected from the group consisting of a shampoo, conditioner, body lotion, skin care composition, deodorant, antiperspirant, after shave lotion, shaving razor, razor blade cartridges, and mixtures thereof. The shaving kit can also comprise a grooming device selected from the group consisting of a cleansing cloth, disposable cleansing cloth (e.g. such as those described in U.S. Pat. Nos. 5,972,361; 5,980,931; 6,060,149; 6,063,397; 6,074,655; 6,132,746; 6,153,208; 6,280,757; 6,338,855; 6,495,151; 6,955,817), a pumice stone, a brush, a comb, a mirror, tweezers and mixtures thereof. The mirror can be a steam free, condensation proof, or non fogging mirror to facilitate shaving in the shower or bath. The components of the shaving kit may be shrink-wrapped together or packaged together in a plastic package.

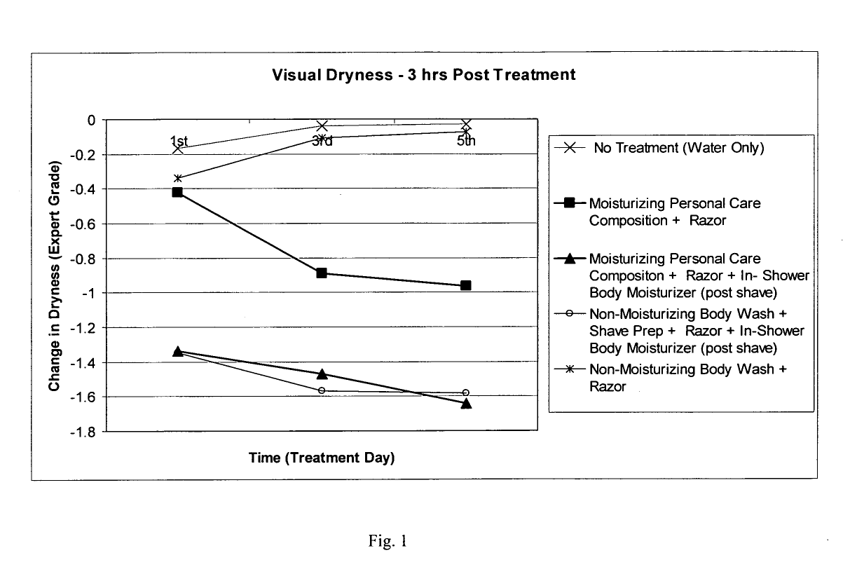

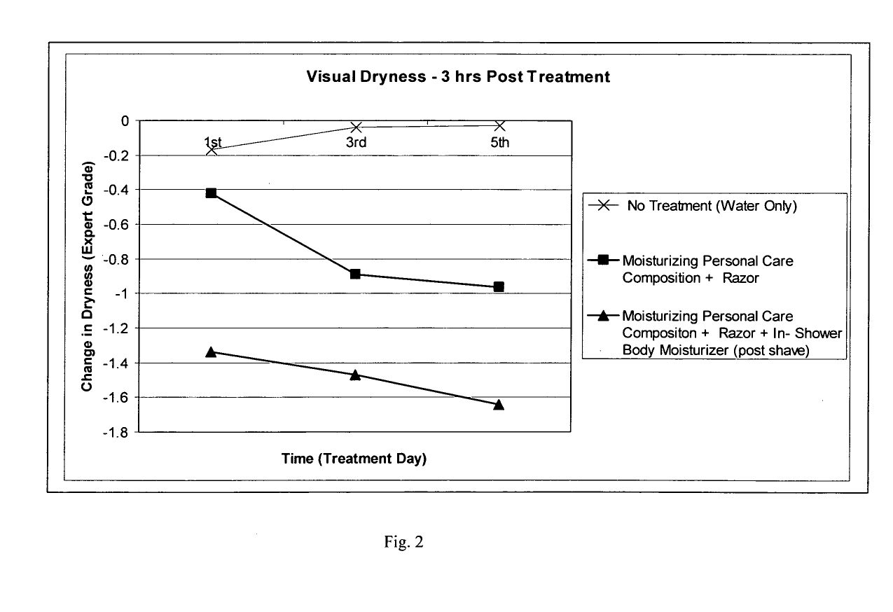

FIGS. 1-8 show the results of a study administered to assess the impact of body wash, shave prep and in-shower body lotion technologies on leg skin condition when used alone or in combination over a one week leg shaving regimen, as described in the clinical method below. FIG. I and FIG. 2 are graphs that plot the change of dryness of skin, as graded by an expert grader, three hours after using various shaving regimes. As shown in FIG. 1 and FIG. 2, the moisturizing personal care composition wash improves dryness better than non-moisturizing body wash and no treatment. The addition of in-shower body lotion to the regime with moisturizing body wash improves dryness better than all other treatments.

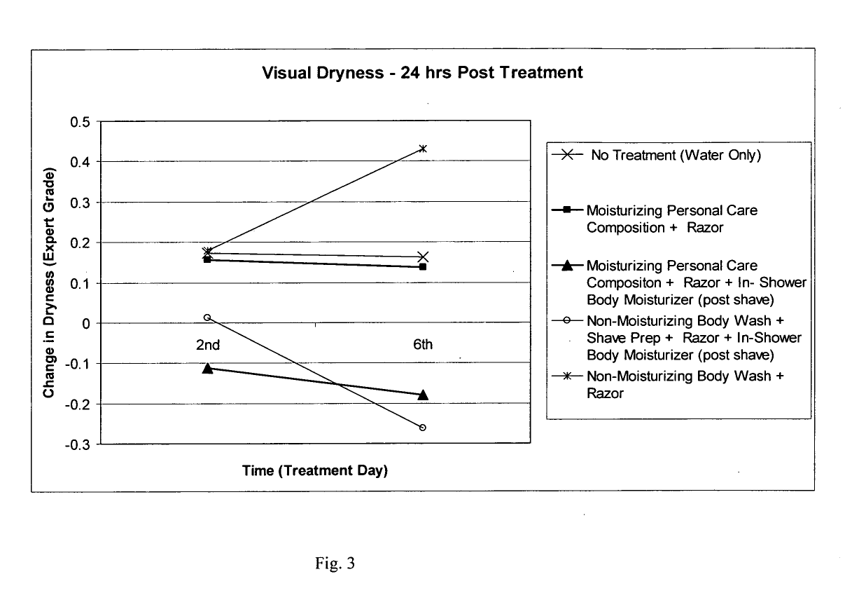

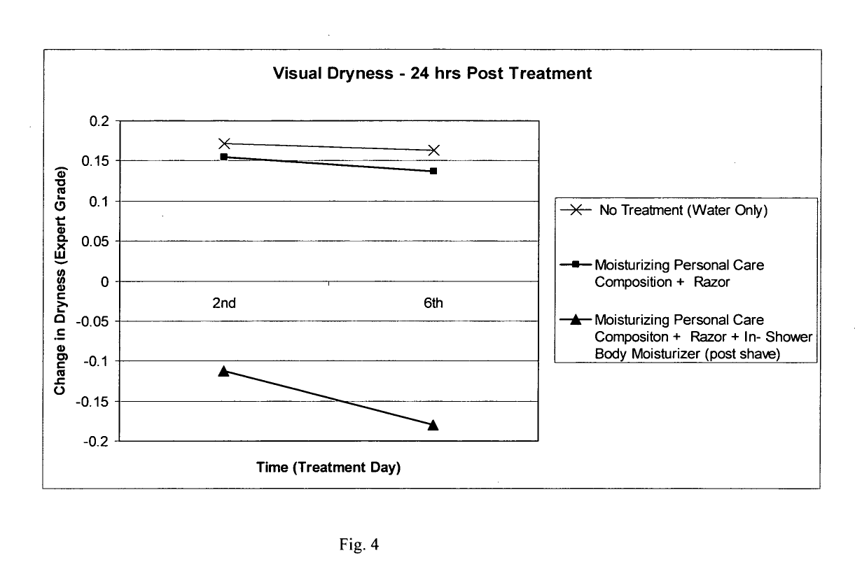

FIG. 3 and FIG. 4 are graphs that plot the change of dryness of skin, as graded by an expert grader, twenty-four hours after using various shaving regimes. As shown in FIG. 3 and FIG. 4, the moisturizing personal care composition improves dryness better than non-moisturizing body wash and no treatment. The addition of in-shower body lotion to the regime with moisturizing body wash improves dryness better than the treatment with moisturizing body wash and razor alone.

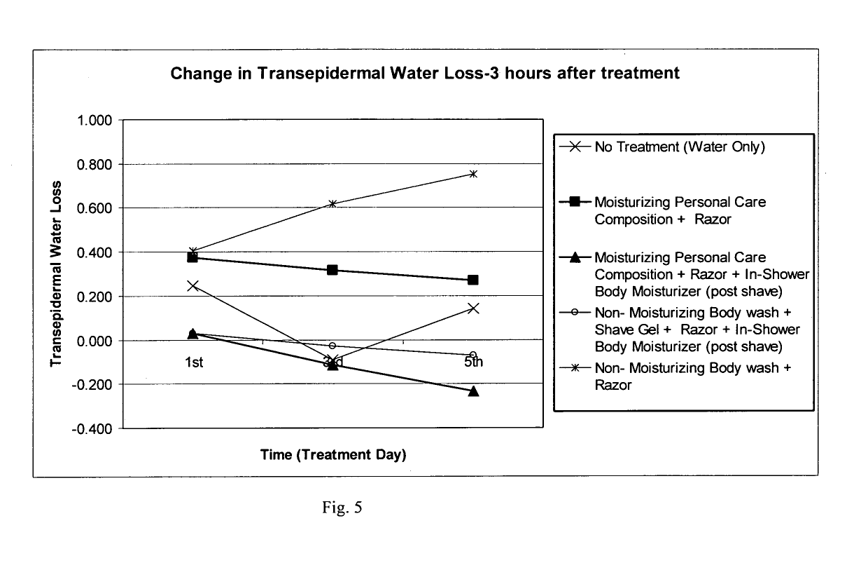

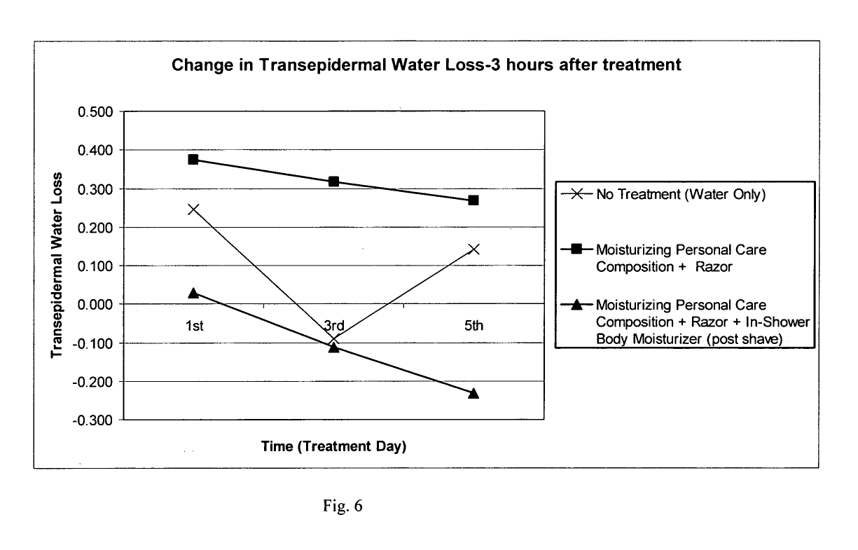

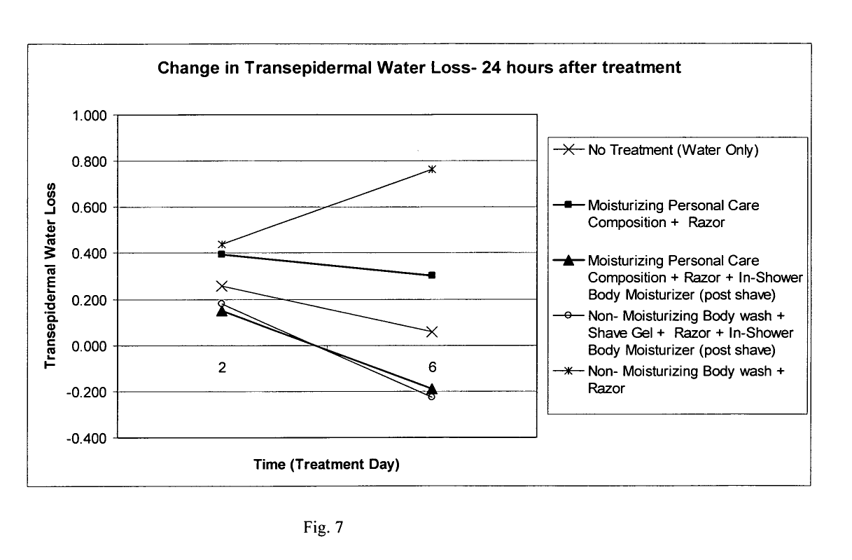

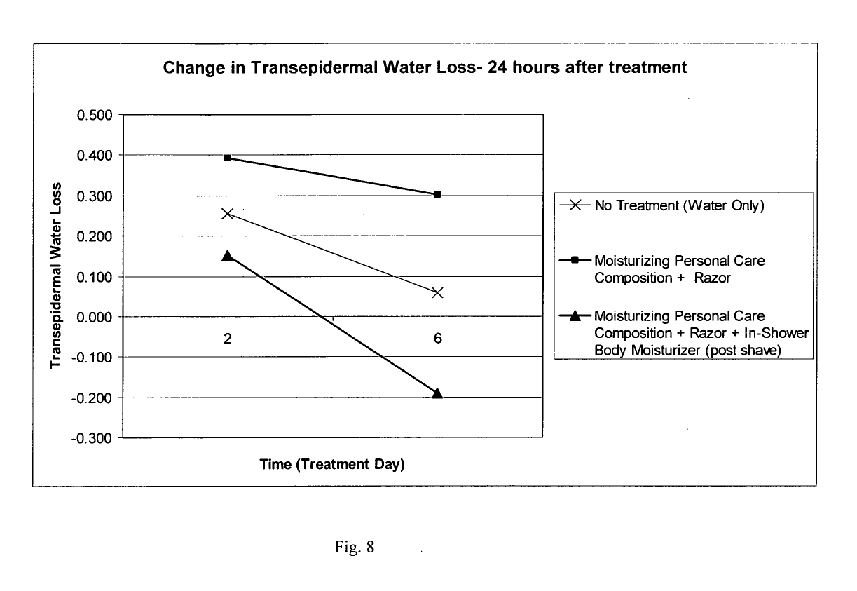

FIG. 5 and FIG. 6 are graphs that plot the change in transepidermal water loss of the skin three hours after various shaving regimes. FIG. 5 and FIG. 6 show the addition of in-shower body lotion to the regime with moisturizing body wash shows less water loss than with moisturizing body wash and razor alone. FIG. 7 and 8 are graphs that plot the change in transepidermal water loss of the skin twenty-four hours after various shaving regimes. The day six data on FIGS. 7-8 (which corresponds to the second data point of each line on these graphs) show an increased moisture loss to skin. The inventors believe that this indicates that part of the stratum corneum was removed reducing the skin's moisture barrier. From the data, it is believed that the addition of in shower body lotion to a shaving regime with moisturizing body wash improves the skin's moisture barrier properties in spite the act of shaving.

### III. Method of Shaving

The present invention relates to a method of shaving, the method comprising the steps of: wetting a skin surface; dispensing the personal care composition from the personal care article comprising a package containing a personal care composition; contacting the skin surface with the personal care composition, and applying a shaving razor to the skin surface. The method further comprises the step of rubbing the personal care composition on the skin to form lather. The method further comprises the step of avoiding the use of a cleansing implement for contacting the skin surface with the personal case composition. The method can further comprise a cleansing step. The cleansing step comprises dispensing the personal care composition onto a cleansing implement and contacting a skin surface with the cleansing implement. The method of shaving is preferably for use in the shower or bath. The method of shaving further comprises the step of rinsing the skin surface of the personal care composition. The method of shaving further comprises the step of dispensing an in-shower body lotion composition from the package of the second personal care article. The method of shaving further comprises the step of contacting the skin surface with the in-shower body lotion composition. The method of shaving further comprises the step of rinsing the skin surface of the in-shower body lotion composition. The method of shaving further comprises the step of drying the skin surface. In another aspect, the personal care composition can be a visually distinct, multiphase personal care composition that comprises a visually distinct cleansing phase comprising a surfactant and water; and a visually distinct benefit phase. The cleansing phase and the benefit phase are in physical contact within the package of the personal care article.

### IV. Personal Care Article

The present invention also relates to a personal care article that comprises an in-shower body lotion composition contained in a package; and a set of instructions in association with the package. The set of instructions comprises instructions to dispense the in-shower body lotion composition from the package, contact skin surface with said in-shower body lotion composition during showering or bathing and after shaving, rinse said skin surface, and dry said skin surface. The package for the personal care article in one aspect is a tottle. The personal care article can further comprise a coupon, rebate, or advertisement. The coupon, rebate or advertisement is associated with one or more of the personal care products selected from the group consisting of a shampoo, conditioner, body lotion, skin care composition, deodorant, antiperspirant, after shave lotion, shaving razor, razor blade cartridge, and mixtures thereof.

### V. Personal Care Composition

The personal care composition can be comprised of a surfactant, water and at least about 15%, by weight of the personal care composition, of a hydrophobic moisturizing agent; at least about 16.5%, by weight of personal care composition, of a hydrophobic moisturizing material, at least about 17%, by weight of personal care composition, of a hydrophobic moisturizing material, 17.5 %, by weight of personal care composition, of a hydrophobic moisturizing material, at least about 20%, by weight of personal care composition, of a hydrophobic moisturizing material, at least about 24%, by weight of personal care composition, of a hydrophobic moisturizing material, at least about 28% hydrophobic moisturizing material; and at least about 30%, by weight of personal care composition, of a hydrophobic moisturizing material. It is believed that adding hydrophobic moisturizing material cushions, lubricates and moisturizes the skin surface for shaving. However, it also believed that personal care compositions with high levels of hydrophobic moisturizing material can cause shaving razors and blades to clog. Thus, the personal care composition of the present invention can comprise less than about 70%, by weight of the personal care composition, of a hydrophobic moisturizing material, preferably less than about 50%, by weight of personal care composition, of a hydrophobic moisturizing material, and even more preferably less than about 40%, by weight of personal care composition, of a hydrophobic moisturizing material. Typically, the personal care composition will comprise from about 15% to about 50%, by weight of the personal care composition, of a hydrophobic moisturizing material, and preferably from about 17% to about 30%, by weight of the personal care composition, of a hydrophobic moisturizing material.

It is believed that the personal care compositions of the present invention would tend to clog typical aerosol containers. Thus, the personal care composition of the present invention will preferably be non-aerosol and will preferably be substantially free of propellants. The personal care composition will preferably comprise less than 1% propellant, more preferably 0.05% propellant, even more preferably 0.01% propellant and most preferably no propellant. These propellants can be any known to one skilled in the art. The personal care compositions of the present invention are substantially free of propellants selected from the group consisting of propane, isobutane and other petroleum distillates, nitrogen, carbon dioxide, dimethylether, methylethylether, methylene chloride, vinyl chloride and fluorochlorohydrocarbons. The latter include Freon 115 pentafluorochloroethane and Freon C-318, octafluorocyclobutane, gaseous chlorofluorinated C₁-C₂ hydrocarbon propellants, and mixtures thereof.

### a. Cleansing Phase

The structured surfactant phase comprises surfactants suitable for application to the skin or hair and which are otherwise compatible with the other essential ingredients in the personal care composition including water. These surfactants include anionic, nonionic, cationic, zwitterionic, amphoteric surfactants, soap, or combinations thereof. The personal care composition comprises from about 5% to about 16%, from about 10% to about 16%, from about 13% to about 15%, by weight of the personal care composition, of lathering surfactants selected from the group consisting of anionic surfactants, nonionic surfactants, amphoteric surfactants, cationic surfactants or mixtures thereof.

Suitable surfactants are described in McCutcheon's, Detergents and Emulsifiers, North American edition (1986), published by allured Publishing Corporation; and McCutcheon's, Functional Materials, North American Edition (1992); and in U.S. Pat. No. 3,929,678 issued to Laughlin, et al on Dec. 30, 1975.

Preferred linear anionic surfactants for use in the structured surfactant phase of the personal care composition include ammonium lauryl sulfate, ammonium laureth sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, potassium lauryl sulfate, and combinations thereof.

Branched anionic surfactants and monomethyl branched anionic surfactants suitable for the present invention are described in a commonly owned, patent application published on Dec., 2006 under U.S. Publication No. 60/680,149 by Smith, et al. Branched anionic surfactants include but are not limited to the following surfactants: sodium trideceth sulfate, sodium tridecyl sulfate, sodium C₁₂₋₁₃ alkyl sulfate, and C₁₂₋₁₃ pareth sulfate and sodium C₁₂₋₁₃ pareth-n sulfate.

In one aspect, the personal care compositions of the present invention may further preferably comprise an amphoteric surfactant, a zwitterionic surfactant and mixtures thereof. In one embodiment, the personal care composition can comprise at least one amphoteric surfactant. Amphoteric surfactants suitable for use in the present invention include those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products described in U.S. Pat. No. 2,528,378. In one aspect, the personal care composition can comprise an amphoteric surfactant that is selected from the group consisting of sodium lauroamphoacetate, sodium cocoamphoactetate, disodium lauroamphoacetate disodium cocodiamphoacetate, and mixtures thereof.

Zwitterionic surfactants suitable for use include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Zwitterionic surfactants suitable for use in the personal care composition include betaines, including cocoamidopropyl betaine.

In some aspects, the personal care composition of the present invention is preferably free of alkyl amines and alkanolamide to ensure mildness of the composition to the skin.

The personal care composition preferably comprises at least one nonionic emulsifier. Preferably the nonionic emulsifier has an HLB from about 1.5 to 13.0, preferably from about 3.4 to 13.0, more preferably 3.4 to about 9.5, more preferably 3.4 to about 8.0. The personal care composition preferably comprises a nonionic emulsifier at concentrations ranging from about 0.1% to about 10%, more preferably from about 0.25% to about 8%, even more preferably from about 0.5% to about 5%, still even more preferably from about 1.0% to about 3%, and still even still more preferably from about 1.5% to about 2.5%, by weight of the personal care compositions.

The balance between the hydrophilic and lipophilic moieties in a surfactant molecule is used as a method of classification (hydrophile-lipophile balance, HLB). The HLB values for commonly-used surfactants are readily available in the literature (e.g., HLB Index in McCutcheon's Emulsifiers and Detergents, MC Publishing Co., 2004). For example, cocamide monoethanolamine (CMEA) is known in the art to have an HLB value of 16.8. Another way of obtaining HLB values is to estimate by calculations. The HLB system was originally devised by Griffin (J. Soc. Cosmetic Chem., 1, 311, 1949). Griffin defined the HLB value of a surfactant as the mol % of the hydrophilic groups divided by 5, where a completely hydrophilic molecule (with no non-polar groups) had an HLB value of 20. Other examples of how to calculate HLB values are described by Davies in Interfacial Phenomena, 2nd Edition, Academic Press, London, 1963 and by Lin in J. Phys. Chem. 76, 2019-2013, 1972. Non-limiting examples of preferred nonionic emulsifiers for use herein are those selected form the group consisting of glyceryl monohydroxystearate, isosteareth-2, trideceth-3, hydroxystearic acid, propylene glycol stearate, PEG-2 stearate, sorbitan monostearate, glyceryl laurate, laureth-2, cocamide monoethanolamine, lauramide monoethanolamine, and mixtures thereof.

An electrolyte can be added per se to the personal care composition or it can be formed in situ via the counterions included in one of the raw materials. The electrolyte includes an anion that comprises phosphate, chloride, sulfate or citrate and a cation that comprises sodium, ammonium, potassium, magnesium or mixtures thereof. Some preferred electrolytes are sodium chloride, ammonium chloride, sodium or ammonium sulfate. The electrolyte is added to the structured surfactant phase in the amount, by weight of the personal care composition, of: from about 0.1% to about 6%, from about 1% to about 5%, from about 2% to about 4%, and from about 3% to about 4%.

### b. Benefit Phase

The personal care compositions of the present invention comprise a benefit phase. The benefit phase in the present invention is preferably anhydrous and can be substantially free of water. The benefit phase can be substantially free or free of surfactant.

The benefit phase typically comprises hydrophobic moisturizing materials. The benefit phase may comprise from about 1% to about 50%, preferably from about 5% to about 30%, more preferably from about 10% to about 30%, by weight of the personal care composition, of a hydrophobic moisturizing material.

Hydrophobic moisturizing materials suitable for use in the present invention preferably have a Vaughan Solubility Parameter of from about 5 (cal/cm³)^{1/2} to about 15 (cal/cm³)^{1/2}, as defined by Vaughan in Cosmetics and Toiletries, Vol. 103. The Vaughan Solubility Parameter (VSP) as used herein is a parameter used to define the solubility of hydrophobic materials. Vaughan Solubility parameters are well known in the various chemical and formulation arts and typically have a range of from 5 to 25. Non-limiting examples of hydrophobic moisturizing materials having VSP values ranging from about 5 to about 15 include the following: Cyclomethicone 5.92, Squalene 6.03, Petrolatum 7.33, Isopropyl Palmitate 7.78, Isopropyl Myristate 8.02, Castor Oil 8.90, Cholesterol 9.55, as reported in Solubility Effects in Product, Package, Penetration and Preservation, C. D. Vaughan, Cosmetics and Toiletries, Vol. 103, October 1988.

The hydrophobic moisturizing materials for use in the benefit phase of the composition have a preferred rheology profile as defined by Consistency value (k) and Shear Index (n). The term “Consistency value” or “k” as used herein is a measure of lipid viscosity and is used in combination with Shear Index, to define viscosity for materials whose viscosity is a function of shear. The measurements are made at 35° C. and the units are poise (equal to 100 cps). The term “Shear Index” or “n” as used herein is a measure of lipid viscosity and is used in combination with Consistency value, to define viscosity for materials whose viscosity is a function of shear. The measurements are made at 35° C. and the units are dimensionless. Consistency value (k) and Shear Index (n) are more fully described in co-pending U.S. application Ser. No. 11/312615 entitled “Shaving Kit, Article of Commerce and Method of Shaving Comprising a Personal Care Composition” filed Dec. 20, 2005. Preferred Consistency value ranges are 1-10,000 poise (1/sec)ⁿ⁻¹, preferably 10-2000 poise (1/sec)ⁿ⁻¹ and more preferably 50-1000 poise (1/sec)ⁿ⁻¹. Shear Index ranges are 0.1-0.8, preferably 0.1-0.5 and more preferably 0.20-0.4. These preferred rheological properties are especially useful in providing the personal cleansing compositions with improved deposition of benefit agents on skin.

The benefit phase can be comprised of the hydrophobic moisturizing materials selected from the group consisting of petrolatum, lanolin, derivatives of lanolin (e.g. lanolin oil, isopropyl lanolate, acetylated lanolin, acetylated lanolin alcohols, lanolin alcohol linoleate, lanolin alcohol riconoleate) hydrocarbon oils (e.g. mineral oil) natural and synthetic waxes (e.g. micro-crystalline waxes, paraffins, ozokerite, lanolin wax, lanolin alcohols, lanolin fatty acids, polyethylene, polybutene, polydecene, pentahydrosqualene) volatile or non-volatile organosiloxanes and their derivatives (e.g. dimethicones, cyclomethicones, alkyl siloxanes, polymethylsiloxanes, methylphenylpolysiloxanes), natural and synthetic triglycerides (e.g. castor oil, soy bean oil, sunflower seed oil, maleated soy bean oil, safflower oil, cotton seed oil, corn oil, walnut oil, peanut oil, olive oil, cod liver oil, almond oil, avocado oil, palm oil, sesame oil) and combinations thereof. In one aspect, at least about 50% by weight of the hydrophobic moisturizing materials are selected from the groups of petrolatum, mineral oil, paraffins, polyethylene, polybutene, polydecene, dimethicones, alkyl siloxanes, cyclomethicones, lanolin, lanolin oil, lanolin wax. The remainder of the hydrophobic moisturizing material can be selected from: isopropyl palmitate, cetyl riconoleate, octyl isononanoate, octyl palmitate, isocetyl stearate, hydroxylated milk glyceride and combinations thereof. The benefit phase of the personal care composition can be comprised a combination of petrolatum and mineral oil.

### VI. In-Shower Body Lotion Composition

The in-shower body lotion compositions of the present invention are selected from the group consisting of skin compatibles oils, high internal phase emulsions, oil in water emulsions, gel networks, non-aqueous systems, and polyerically stabilized dispersions and mixtures thereof. Examples of in-shower body lotion compositions are described in detail in U.S. Publication No. 2004/0223992, U.S Publication No. 2003/0190296, U.S Publication No. 2003/0054019, U.S Publication No. 2003/0049282, U.S. Pat. No. 6,699,488 and U.S. Pat. No. 6,645,511.

The in-shower body lotion can comprise a skin compatible oil. In one aspect, the in-shower body lotion comprises a skin compatible oil at levels by weight of the in-shower body lotion composition of: less than about 80%, less than about 70%; less than about 60% and less than about 50%. In one aspect, the in-shower body lotion comprises a skin compatible oil by weight of the total in-shower body lotion composition at levels of: at least about 1%, at least about 5%, at least about 7%, and at least about 10%.

The in-shower body lotion can comprise an oil in water emulsion that comprises a skin compatible oil and an aqueous phase. In the preferred embodiment the oil in water emulsion comprises a continuous aqueous phase, a discontinuous structured oil phase, and an aqueous phase stability agent. In one aspect, these oil and water emulsions are effectively ‘non-lathering’. In one aspect, the in-shower body lotion can comprise less than about 2%, less than about 1.5%, less than about 1.0%, and less than about 0.5%, by weight of the in-shower body lotion composition, of an anionic surfactant.

The aqueous phase of a oil in water emulsion is the continuous phase in which the structured oil phase is dispersed. The aqueous phase can also comprise a stability agent, preservatives, wetting agents, auxiliary emulsifiers and optional benefit agents. The continuous aqueous phase comprises a fluid at levels by weight of the in-shower body lotion of: less than about 90%, less than about 80%, less than about 70%, and less than about 60%. The continuous aqueous phase comprises a fluid at levels by weight of the in-shower body lotion of: at least 10%, at least 20%, at least 30%, and at least 40%.

The structured oil phase of an oil in water emulsion comprises two essential components: a skin compatible oil and a structurant. The structurant must satisfy two requirements. First, the structurant must be capable of forming a stable network in the skin compatible oil phase at a temperature below 35° C., so that the structured oil is active during use, but is not perceived as gritty. By stable network, we mean the network survives at least one month of storage at 25° C. and 35° C. The second requirement is that the structurant provides the structured oil phase with the correct rhelogical properties. The structured oil phase should have a viscosity in the range of 100 to about 200,000 poise measured at 1 Sec-1, in one aspect 200 to about 100,000 poise, and most preferably 200 to about 50,000 poise as determined using the lipid rheology method described in U.S. Publication No. 2004/0223992. The amount of structurant required to produce this viscosity will vary depending on the type of oil and the type of structurant. In general, the amount of structurant will be less than about 75%, less than about 50%, and less than about 35%, by weight of the structured oil phase. Structurants meeting the above requirements can form a 3-dimensional network to build up the viscosity of the skin compatible oil. It has been found that such structured oil phases with the 3-dimensional network, are extremely desirable for use as wet-skin treatment compositions used in bathing. It is believed that structured oil phases have an in-use property of depositing effectively on skin and have the after use properties of being retained effectively on wet skin even after rinsing and drying to provide long-lasting after wash skin benefits without causing a too oily/greasy wet and dry feel. It is believed that these in-use and after-use properties are due to the shear thinning rheological properties and the weak structure of the network. Due to its high low-shear viscosity, structured oil can stick and retain well on the skin during application. After being deposited on the skin, the network yields easily during rubbing due to the weak structuring of the crystal network and its lower high-shear viscosity.

The degree of shear-thinning (which is described in the Lipid Rheology Method described in U.S. Publication No. 2004/0223992) exhibited by the structured oil phase is given by the value of n from the Power Law Model. For the present invention, it is preferred that the structured oil phase have a shear index less than 0.8, more preferably less than 0.6, even more preferably less than 0.5 and most preferably less than 0.4.

The structurants can be an organic structurant that is either crystalline solids or amorphous gels with molecular weight less than 5,000 Daltons, preferably less than 3,000 Daltons. Preferred organic structurants have a melting point greater than 35° C., preferably greater than 40° C. Especially preferred structurants are those that can form a solution with the selected skin compatible oil at a temperature higher than their melting point to form a free flowing clear solution. Upon cooling to the ambient temperature, the organic structurant precipitate from the oil phase to form a 3-dimensional structure providing the physical properties set forth above. Examples of organic thickeners suitable for the invention are solid fatty acid esters, natural or modified fats, fatty acid, fatty amine, fatty alcohol, natural and synthetic waxes, and petrolatum. Examples of these structurants are further described in U.S. Publication No. 2004/0223992, U.S Publication No. 2003/0190296, U.S Publication No. 2003/0054019, U.S Publication No. 2003/0049282, U.S. Pat. No. 6,699,488 and U.S. Pat. No. 6,645,511.

In-shower body lotion can comprise one or more aqueous phase stability agent. The aqueous phase stability agent is a polymeric stabilizer. In one aspect, the in-shower body lotion can comprise levels of polymeric stabilizer, by weight of the in-shower body lotion, of less than about 10%, less than 8%, and less than 7%. In one aspect, the in-shower body lotion can comprise levels of polymeric stabilizer, by weight of the in-shower body lotion, of: at least 0.01%, at least 0.05%, and at least 0.1%. It is believed that polymer stabilizer builds viscosity of the in-shower body lotion. This can be measured using the Polymeric Stabilizer Viscosity Test as described in U.S. Publication No. 2004/0223992. Preferably, the stability agent produces a viscosity in this test of at least 1000 cps, more preferably at least 1500 cps, and still more preferably at least 2000 cps. Non-limiting examples of polymeric stabilizers useful herein include carboxylic acid polymers, polyacrylamide polymers, crosslinked polyacrylate polymers, polysaccharides, gums, modified starches. Examples of these polymeric stabilizers are further described in U.S. Publication No. 2004/0223992 and U.S. Pat. No. 6,699,488

The in-shower body lotion can comprise a gel comprising a hydrophobic structuring agent and a non-ionic, hydrophilic surfactant. Without being bound by theory, it is believed that the gel-network allows for good application of the product in the wet environment of bathing, allowing the product to be spread easily and deposited evenly on the skin of the user. Higher levels of gel-network interfere with deposition, effectively releasing fewer lipids from the composition and resulting more lipid rinse-off lowering deposition efficiency. Without a gel-network in the product, the product would consist essentially of water, an aqueous phase stability agent and lipid, thus the application characteristics of the product would be undesirable in that the product would be difficult to spread and deposit evenly. -For this reason, when the gel-network is present the level of gel-network is kept relatively low. In a preferred embodiment, the gel-network will not form a homogeneous aqueous phase as described in the gel-network stability test described in U.S. Publication No. 2004/0223992.

Hydrophobic structuring agent is one component of the gel-network. The hydrophobic, structuring agent can be selected from the group consisting of saturated C₁₆ to C₃₀ fatty alcohols, saturated C₁₆ to C₃₀ fatty alcohols containing from about 1 to about 5 moles of ethylene oxide, saturated C₁₆ to C₃₀diols, saturated C₁₆ to C₃₀ monoglycerol ethers, saturated C₁₆ to C₃₀ hydroxy fatty acids, and mixtures thereof, having a melting point of at least about 40° C. In one aspect, the in-shower body lotion comprises a hydrophobic structuring agent at levels, by weight of the in-shower body lotion, of: less than about 20%, less than 10%, and less than 5%. In one aspect, the in-shower body lotion comprises a hydrophobic structuring agent at levels by weight of the in-shower body lotion of: at least 1%, at least 2%, and at least 3%. Without being limited by theory, it is believed that these structuring agents are useful to assist in the formation of the rheological characteristic of the composition which contributes to the hydrolytic stability of the composition of the present invention. In particular, structuring agents assist in the formation of the liquid crystalline gel-network structures.

The preferred structuring agents of the present invention are selected from the group consisting of stearyl alcohol, cetyl alcohol, behenyl alcohol, stearic acid, palmitic acid, the polyethylene glycol ether of stearyl alcohol having an average of about 1 to about 5 ethylene oxide units, the polyethylene glycol ether of cetyl alcohol having an average of about 1 to about 5 ethylene oxide units, the polyethylene glycol ether of stearyl alcohol having an average of about 2 ethylene oxide units (steareth-2), the polyethylene glycol ether of cetyl alcohol having an average of about 2 ethylene oxide units, steareth-2 and mixtures thereof.

Hydrophilic surfactant is one component of the gel-network. The surfactant, at a minimum, must be hydrophilic enough to disperse in water. Without being limited by theory, it is believed that the hydrophilic surfactant disperses the hydrophobic materials (e.g. the structuring agent), in the water phase. The exact surfactant chosen will depend upon the pH of the composition and the other components present. One example is nonionic surfactants further described in application U.S. Publication No. 2004/0223992. In one aspect, the in-shower body lotion comprises a hydrophilic surfactant at levels, by weight of the in-shower body lotion, of less than about 10%, less than about 6%, and less than about 3%. In one aspect, the in-shower body lotion comprises a hydrophilic surfactant at levels, by weight of the in-shower body lotion, of: at least 0.1%, at least 0.2%, and at least 0.3%.

Emulsifiers can be useful in some embodiments of the in-shower body lotion. Nonlimiting examples include PROLIPID 141 (glyceryl stearate, behenyl alcohol, palmitic acid, stearic acid, lecithin, lauryl alcohol, myristyl alcohol and cetyl alcohol) and 151 (Glyceryl stearate, cetearyl alcohol, stearic acid, 1-propanamium, 3-amino-N-(2-(hydroxyethyl)-N-N-Dimethyl,N-C(16-18) Acyl Derivatives, Chlorides) from ISP; POLAWAX NF (Emulsifying wax NF), from Croda; and EMULLIUM DELTA (cetyl alcohol, glyceryl stearate, peg-75 stearate, ceteth-20 and steareth-20) from Gattefosse.

The in-shower body lotion can comprise one or more benefit agents, as described above or one or more optional ingredients, as described below. Preferably when the in-shower body lotion composition comprises an oil in water emulsion, the benefit agent is dispersed within the skin compatible oil. When the oil in water emulsion comprises a structured oil phase the benefit agent is preferably dispersed within the structured oil phase.

### VII. Optional Ingredients

While not essential for the purposes of the present invention, the non-limiting list of optional materials, illustrated hereinafter are suitable for use in personal care compositions, and may be incorporated in certain embodiments, for example to assist or enhance cleansing performance, for treatment of the skin, or to modify the aesthetics of the personal care composition. Optional materials useful in the products herein are described by their cosmetic and/or therapeutic benefit or their postulated mode of action or function. These descriptions are non-limiting and made for the sake of convenience because it is understood that these materials can provide more than one benefit, function or operate via more than one mode of action. The precise nature of these optional materials, and levels of incorporation thereof, will depend on the physical form of the composition and the nature of the cleansing operation for which it is to be used. The amount of optional materials in compositions are usually formulated, by weight of the composition, at less than about less than about 6%, less than about 5%, less than about 4%, less than about 3%, less than about 2%, less than about 1%, less than about 0.5%, less than about 0.25%, less than about 0.1%, less than about 0.01%, less than about 0.005%.

Optional ingredients, which can be used in the personal care compositions of the present invention, can be selected from the group consisting of thickening agents; low density microspheres (e.g. Expancel 091 WE40 d24, Akzo Nobel and others described in commonly owned and assigned U.S. Patent Publication No. 2004/0092415A1 published on May 13, 2004); preservatives; antimicrobials; fragrances; chelators (e.g. such as those described in U.S. Pat. No. 5,487,884 issued to Bisset, et al.); sequestrants; vitamins (e.g. Retinol); vitamin derivatives (e.g. tocophenyl actetate, niacinamide, panthenol); sunscreens; desquamation actives (e.g. such as those described in U.S. Pat. Nos. 5,681,852 and 5,652,228 issued to Bisset); anti-wrinkle/ anti-atrophy actives (e.g. N-acetyl derivatives, thiols, hydroxyl acids, phenol); anti-oxidants (e.g. ascorbic acid derivatives, tocophenol) skin soothing agents/skin healing agents (e.g. panthenoic acid derivatives, aloe vera, allantoin); skin lightening agents (e.g. kojic acid, arbutin, ascorbic acid derivatives) skin tanning agents (e.g. dihydroxyacteone); polymeric phase structurant (e.g. naturally derived polymers, synthetic polymers, crosslinked polymers, block copolymers, copolymers, hydrophilic polymers, nonionic polymers, anionic polymers, hydrophobic polymers, hydrophobically modified polymers, associative polymers, and oligomers); a liquid crystalline phase inducing structurant (e.g. trihydroxystearin available from Rheox, Inc. under the trade name THIXCIN® R); organic cationic deposition polymer (e.g. Polyquaternium 10 available from Amerchol Corp. Edison, N.J., USA, guar hydroxypropyltrimonium chloride available as Jaguar C-17 from Rhodia Inc., and N-Hance polymer series commercially available from Aqualon); anti-acne medicaments; essential oils; sensates; pigments; colorants; pearlescent agents; interference pigments (e.g such as those disclosed in U.S. Pat. No. 6,395,691 issued to Liang Sheng Tsaur, U.S. Pat. No. 6,645,511 issued to Aronson, et al., U.S. Pat. No. 6,759,376 issued to Zhang, et al, U.S. Pat. No. 6,780,826 issued to Zhang, et al.) particles (e.g. talc, kolin, mica, smectite clay, cellulose powder, polysiloxane, silicas, carbonates, titanium dioxide, polyethylene beads) hydrophobically modified non-platelet particles (e.g. hydrophobically modified titanium dioxide and other materials described in a commonly owned, patent application published on Aug. 17, 2006 under Publication No. 2006/0182699A by Taylor, et al.) and mixtures thereof. Other optional ingredients are most typically those materials approved for use in cosmetics and that are described in the CTFA Cosmetic Ingredient Handbook, Second Edition, The Cosmetic, Toiletries, and Fragrance Association, Inc. 1988, 1992.

### VIII. Method of Manufacturing

The personal care compositions of the present invention may be prepared by any known or otherwise effective technique, suitable for making and formulating the desired product form. The visually distinct, multiphase form can be made by combining toothpaste-tube filling technology with a spinning stage design, the method and apparatus as disclosed in U.S. Pat. No. 6,213,166 issued to Thibiant, et al and the method disclosed in commonly owned patent application published on Nov. 18, 2004 under U.S. Publication No. 2004/0219119 A1 by Wei, et al.

### IX. Clinical Method

A study was administered to assess the impact of body wash, shave prep and in-shower body lotion technologies on leg skin condition when used alone or in combination over a one week leg shaving regimen. The various leg shaving regimens and an untreated control were tested in a blinded, randomized study that followed a protocol based on a modification of published procedures that are used to assess the dry skin improvement potential of in-shower personal care products (Ertel, K. D., Neumann, P. B., Hartwig, P. M., Rains, G. Y., and Keswick, B. H., Leg Wash protocol to assess the skin moisturization potential of personal cleansing products. Int. J. Cosmet. Sci. 21: 383-397 (1999).

The study was conducted over 13 consecutive days, including a 7-day reconditioning phase, a 5-day treatment phase and a 1 day regression phase. The randomized study employed an incomplete block design in which 4 of the 7 treatments was evaluated on each subject, 2 treatments on each leg. After the 7-day preconditioning phase, subjects returned to the test facility to have the skin on their lower legs evaluated by an expert grader. Only subjects that exhibited sufficient dryness on all of the treatment sites will qualify to continue into the treatment phase.

Healthy adult females used Olay® bar soap for bathing and showering during a 7-day preconditioning treatment period. During preconditioning, enrolled subjects used an OLAY® bar with no exfoliating beads provided by the test facility in place of their usual product(s) for bathing and showering. Moisturizer use and leg shaving were restricted.

Each subject's lower legs were visually evaluated by a qualified expert grader for dryness and redness at baseline (prior to the first treatment). The grader determined the qualification of the subjects tested. A subject must not have cuts, scratches, rashes, or any condition (e.g., excessive hair growth, extensive scarring, birth marks, acne, port wine stains, and tattoos) on her legs that might interfere with the visual or instrumental assessments. A subject must exhibit visual dryness grades ≧2.0 and <4.0 (0.0 to 6.0 scales) on each treatment site on the outer surface of both lower legs to qualify. The visual dryness grading scale used by the expert graders are described below in Table 1.

TABLE 1
Visual Dryness Grading Scales

| Grade^{a} | Dryness^{b} | Redness |
|---|---|---|
| 0.0 | perfect skin | no redness |
| 1.0 | patches of checking and/or slight powderiness, | barely |
| | occasional | detectable |
| | | redness |
| 2.0 | generalized slight powderiness, early cracking | slight redness |
| | or occasional | |
| 3.0 | generalized moderate powderiness and/or | moderate |
| | moderate cracking | redness |
| 4.0 | generalized heavy powderiness and/or heavy | heavy or |
| | cracking and | substantial |
| | | redness |
| 5.0 | generalized high cracking and lifting scales, | severe redness |
| | eczematous change | |
| 6.0 | generalized severe cracking, bleeding cracks | extreme |
| | and eczematous changes may be present, large | redness |
| | scales may be sloughing off | |

^{a}half-unit grades may be used if necessary
^{b}‘generalized’ refers to situations where more than 50% of the application area is affected

After pre-conditioning and qualification, subjects with sufficient dry leg skin entered a 6 day treatment period. During the treatment period, technicians washed subjects' legs with the assigned cleanser following a prescribed procedure, as detailed below. In-shower body lotion was applied to designated sites after rinsing. On alternate days, beginning with day 1, subjects used a provided razor to shave each washed site following a prescribed procedure before rinsing. Legs were then rinsed and in-shower body lotion was applied, as appropriate.

The seven treatments included: Olay® bar soap and Venus® razor; Olay® Purifying Body Wash and Venus® razor; Olay® Body Wash plus Créme Ribbons and Venus® razor; Olay® Purifying Body Wash, Gillette® Satin Care Shave Gel and Venus® razor; Olay® Body Wash plus Créme Ribbons, Venus® razor and Olay® Ultra Moisture In-Shower Body Lotion with Shea Butter (post shave); Olay® Purifying Body Wash, Gillette® Satin Care Shave Gel, Venus® razor and Olay® Ultra Moisture In-Shower Body Lotion with Shea Butter (post shave); and a No Treatment Control. All body wash products will be applied with treated puffs.

Before initially grading the subjects' legs on Study Day 8, test a technician marked off the leg application areas. Each leg was marked with two 70 cm² areas measuring 7 cm across and 10 cm on the outer aspect of the subjects' lower legs using a template and laboratory marking pen (corner brackets are sufficient to delineate each area). As well, water flow rate measurements were made daily prior to the first subject wash, the water flow rate should be 1,200 ml per minute.

During the treatment period, trained technicians treat each subject's legs. The technician began with the upper site on the left leg. During the first step, the technician wetted the treatment area for 5 seconds with 95-100° F. (35-37.8° C.) running tap water. During the second step, the test product was applied to the assigned site by the technician, using the appropriate procedure according to product, as shown in Table 2. During the third step, the lather remained on the application site for 90 seconds. After the residence time for a particular application site has expired, the technician proceeded to the fourth step and rinsed the site for 15 seconds under a running tap, taking care not to rinse the adjacent sites. After the application area has been rinsed, the technician gently patted the area dry with a disposable paper towel and was careful not to rub the skin. Using the appropriate treatments, this entire procedure was repeated on the lower site on the left leg before conducting the entire procedure on both sites of the right leg.

TABLE 2
Treatment Procedure

| Product Type | Procedure |
|---|---|
| “No Treatment” | No product is applied to this site. Wait approximately 10 seconds. Go to |
| (water only) | fourth step recited above. |
| Bar | Wet the bar and both gloved hands under the running tap. Roll the bar in |
| | both hands for 6 full rotations. (2 bar flops = 1 full rotation) Scrape all of |
| | the lather generated into the hand to be used for washing. Gently rub the |
| | lathered hand up and down within the marked treatment area for 10 |
| | seconds. Go to third step recited above. |
| Personal Care | While holding the puff in one hand, wet the puff for 5 seconds under the |
| Compositions (e.g. | running tap, then allow the excess water to drain off the puff for 10 |
| Body washes) | seconds without shaking or squeezing the puff. Dispense 0.7 ml of body |
| | wash product from the syringe onto the center of the treatment area. Place |
| | the wet puff over the dispensed product and gently rub the puff back and |
| | forth within the appropriate site for 10 seconds. Go to third step recited |
| | above. |
| Personal Care | Following the body wash product procedure proceed with lotion |
| Compositions (e.g. | application per assigned regimen for this site. Dispense 70 μl of the |
| Body washes) + In- | lotion component to the wet treatment area. Gently rub the product for 20 |
| Shower Body | seconds covering the entire treatment area. The lotion remains on the |
| Lotion | treatment site for 40 seconds. Once the residence time has expired, rinse |
| | the site for 10 seconds. Gently pat the treatment site dry with a disposable |
| | paper towel. |

After the first, third and fifth treatments, the technician begins with upper site on the left leg site and applies the test product assigned to that site, using the appropriate procedure according to product type, as shown in Table 3. The subject is asked to shave the marked site with a wetted razor by stroking in an upward motion over the site 4 times, with the first stroke beginning near the shin. The technician rinsed the site for 15 seconds under a running tap, taking care not to rinse the adjacent sites. After the application area was rinsed, the technician gently patted the area dry with a disposable paper towel and was careful not to rub the skin. Using the appropriate treatments, this entire procedure was repeated on the lower site on the left leg before conducting the entire procedure on both sites of the right leg.

TABLE 3
Shaving Procedure after the 1^{st}, 3^{rd} and 5^{th} treatment

| Product Type | Procedure |
|---|---|
| Bar | Following the bar product treatment procedure proceed with shaving |
| | procedure per assigned regimen for this site. Hand the subject the razor. |
| | Wet the bar and both gloved hands under the running tap. Roll the bar in |
| | both hands for 6 full rotations. (2 bar flops = 1 full rotation) Scrape all |
| | of the lather generated into the hand to be used for washing. Gently rub |
| | the lathered hand up and down within the marked treatment area for 10 |
| | seconds. Tell subject to wet razor for 5 seconds and begin the shaving |
| | site, as recited above. |
| Personal Care | Following the body wash product treatment procedure proceed with |
| Compositions (e.g. | shaving procedure per assigned regimen for these sites. Hand the |
| Body washes) | subject the razor. While holding the appropriately labeled puff in one |
| | hand, wet the puff for 5 seconds under the running tap, then allow the |
| | excess water to drain off the puff for 10 seconds without shaking or |
| | squeezing the puff. Dispense 0.7 ml of body wash product from the |
| | syringe onto the center of the treatment area. Place the wet puff over the |
| | dispensed product and gently rub the puff back and forth within the |
| | appropriate site for 10 seconds. Tell subject to wet razor for 5 seconds |
| | and begin shaving the site as, recited above. |
| Shave Preparations | Following the body wash product treatment procedure proceed with |
| (e.g. Shave Gels) | shaving procedure per assigned regimen for this site. Weigh out 0.7 g |
| | shave prep product into a clean weigh boat just prior to application |
| | (0.65-0.75 is acceptable) Hand the subject the appropriately labeled |
| | razor. Wet gloved fingers under running water and then scoop the 0.7 g |
| | dose of product from weigh boat directly onto the treatment using the |
| | wetted gloved fingers. Gently rub the shave prep product up and down |
| | within the marked treatment area with the wetted gloved fingers for 10 |
| | seconds. Tell subject to wet razor for 5 seconds and begin shaving the |
| | site, as recited above. |
| In-Shower Body | Following the shaving procedure proceed with lotion application per |
| Lotions | assigned regimen for these sites. Dispense 70 μl of the lotion component |
| | to the wet treatment area. Gently rub the product for 20 seconds |
| | covering the entire treatment area. The lotion remains on the treatment |
| | site for 40 seconds. Once the residence time has expired, rinse the site |
| | for 10 seconds. Gently pat the treatment site dry with a disposable paper |
| | towel. DO NOT RUB! |

The subjects will be provided puffs for use in application of the body washes. The technician while holding the appropriately labeled puff in one hand, prepared the puff by wetting the puff for 5 seconds under the running tap and the excess water was drained off the puff for 10 seconds without shaking or squeezing the puff. The technician dispensed 9.3 ml of appropriate body wash product onto the puff in a broad circular pattern. The technician held the puff in one hand the puff was squeezed until the technician just felt the core of the puff. During the wash, the technician does 10 rotations forward alternating hands, then repeat in the opposite direction for 10 rotations alternating hands, for a total of 20 rotations. Following the wash, the technician held the puff in one hand, rinsed the puff for 20 seconds under the running tap, then allowed the excess water to drain off then hang to dry. All of the puffs will be treated with 9.3 ml of the appropriate treatment each day after treatment.

The in-shower body lotion will be dispensed at 1 μl/cm² (70 μl) for each 7 cm×10 cm site. All body wash products will be dispensed at 0.7 ml (dose targeted at 10 μl/cm²). The in-shower body lotion will be mixed then be drawn into syringes at the 0.7 ml dosage. Shave prep products will be weighed out at 0.7 g (0.65-0.75 g is acceptable) (dose targeted at 10 μg/cm²) in a clean weigh boat just prior to application.

At each evaluation, subjects will acclimate for a minimum of 30 minutes in a room with the environment maintained at 70° F. (21.1° C.)±2 and 30-45% relative humidity prior to visual grading and non-invasive instrumental measurements being made on their legs. All evaluations will be made in the controlled environment described above. The evaluations include expert grading of visual dryness, as described above and stratum corneum barrier function. The evaluations were completed at defined times during the treatment period, including: Study Day 8—approximately 3 hours post treatment #1; Study Day 9—approximately 24 hours post treatment #1; Study Day 10—approximately 3 hours post treatment #3; Study Day 12—approximately 3 hours post treatment #5; Study Days 13—approximately 24 hours post treatment #5.

Stratum corneum barrier function or transepidermal water loss (TEWL) was measured at these times. Transepidermal water loss (TEWL) is a measure of cutaneous barrier function and also reflects skin water content. Trans epidermal water loss (TEWL) was measured with the DermaLab® Evaporimeter equipped with dual probes. Each measurement consists of readings collected for 60 seconds with the mean of the last 20 seconds recorded from both probes. These measurements will be made according to published manufacturer's guidelines.

### X. EXAMPLES

The following examples described in Table 4 and 5 are non-limiting examples of personal care compositions of the first personal care article of the present invention. All exemplified amounts are concentrations by weight of the total personal care composition, unless otherwise specified.

TABLE 4
Personal Care Composition of the Present Invention

| | Example # | | |
|---|---|---|---|
| Ingredient (by weight %) | 4 | 5 | 6 |
| I. Cleansing Phase Composition | | | |
| Sodium Trideceth Sulfate^{1.} | 8.1 | 8.1 | 8.1 |
| Sodium Lauryl Sulfate | 8.1 | 8.1 | 8.1 |
| Sodium Lauroamphoacetate^{2.} | 4.8 | 4.8 | 4.8 |
| Guar Hydroxypropyltrimonium Chloride^{3.} | 0.6 | 0.6 | 0.6 |
| PEG 90M^{4.} | 0.15 | 0.15 | 0.15 |
| Isosteareth-2^{5.} | — | 1 | 2 |
| Trideceth-3^{6.} | 2 | 1 | — |
| Xanthan Gum^{7.} | 0.22 | 0.22 | 0.22 |
| Sodium Chloride | 4.75 | 4.75 | 4.75 |
| Disodium EDTA^{8.} | 0.15 | 0.15 | 0.15 |
| Sodium Benzoate | 0.2 | 0.2 | 0.2 |
| Methyl chloro isothiazolinone & methyl | 0.03 | 0.03 | 0.03 |
| isothiazolinone^{9.} | | | |
| Perfume | 1.4 | 1.9 | 1.3 |
| Hollow microspheres^{10.} | 0.33 | 0.33 | 0.33 |
| Water | Q.S. | Q.S. | Q.S. |
| pH adjust to (use citric acid or NaOH) | 6.0 | 6.0 | 6.0 |
| II. Benefit phase Composition | | | |
| G2218 Petrolatum^{11.} | — | — | 70 |
| Petrolatum^{12.} | — | 60 | — |
| Petrolatum¹³. | 99.99 | — | — |
| Mineral Oil^{14.} | — | 39.99 | 29.99 |
| Red 7 Cosmetic Pigment | 0.01 | 0.01 | 0.01 |
| Cleansing Phase:Benefit Phase Ratio | 75:25 | 65:35 | 80:20 |

^{1.}Cedepal TD-407 available from Stepan;
^{2.}Miranol L-32 available from Rhodia;
^{3.}N-Hance 3196 polymer available from Aqualon;
^{4.}Polyox WSR 301 available from Dow Chemical;
^{5.}available from Global Seven, Franklin, NJ;
^{6.}Iconal TDA-3 from BASF Corp.;
^{7.}Keltrol 1000, CP Kelco;
^{8.}Dissolvine NA 2x;
^{9.}Kathon CG, Rohm & Haas;
^{10.}Expancel 091 WE40 d24, Akzo Nobel;
^{11.}available WITCO;
^{12.}Superwhite Protopet, available from WITCO;
¹³.available from Quidesa, Mexico;
^{14.}Hydrobrite 1000 White Mineral Oil.

Prepare the compositions described above by conventional formulation and mixing techniques. Prepare the cleansing phase by preparing a citric acid premix by adding citric acid into water at 1:3 ratio and a Polyox premix by adding Polyox WSR 301 and Keltrol 1000 to Trideceth-3 and Isosteareth-2. Then, add the following ingredients into the main mixing vessel with agitation: water, N-Hance 3196, Expancel, and Polyox premix. Then, add sodium trideceth sulfate, sodium lauroamphoacetate, sodium lauryl sulfate. Add sodium chloride, disodium EDTA, sodium benzoate, and Kathon CG. Adjust pH to 6.0 by adding citric acid premix. Then, add perfume and keep mixing until homogeneous. Prepare the benefit phase by adding petrolatum into a mixing vessel. Heat the vessel to 190° F. Then, add mineral oil with agitation. Add cosmetic pigment and let the vessel cool down with slow agitation.

The cleansing and benefit phases are density matched to within 0.05 g/cm³. Package both phases at specified cleansing phase to benefit phase ratio using one of the methods of manufacturing described herein. The sample stage spins the bottle during filling process to create a marbled appearance.

TABLE 5
Personal Care Composition of the Present Invention

| | Example # | | |
|---|---|---|---|
| Ingredient (by weight %) | 7 | 8 | 9 |
| Sodium Trideceth Sulfate^{1.} | 6.1 | 5.3 | 6.9 |
| Sodium Lauryl Sulfate | 6.1 | 5.3 | 6.9 |
| Sodium Lauroamphoacetate^{2.} | 3.6 | 3.1 | 4.1 |
| Guar Hydroxypropyltrimonium Chloride^{3.} | 0.45 | 0.39 | 0.51 |
| PEG 90M^{4.} | 0.11 | 0.1 | 0.13 |
| Trideceth-3^{5.} | 1.5 | 1.3 | 1.7 |
| Xanthan Gum^{6.} | 0.16 | 0.14 | 0.19 |
| Sodium Chloride | 3.5 | 3.1 | 4.0 |
| Disodium EDTA^{7.} | 0.11 | 0.1 | 0.13 |
| Sodium Benzoate | 0.15 | 0.13 | 0.17 |
| Methyl chloro isothiazolinone & methyl | 0.02 | 0.02 | 0.03 |
| isothiazolinone^{8.} | | | |
| Perfume | 1.1 | 1.1 | 1.1 |
| Hollow Microspheres^{9.} | 0.25 | 0.21 | 0.28 |
| Petrolatum^{10.} | 25 | 35 | 15 |
| Water | Q.S. | Q.S. | Q.S. |
| pH adjust to (use citric acid or NaOH) | 6.0 | 6.0 | 6.0 |

^{1.}Cedepal TD-407 available from Stepan;
^{2.}Miranol L-32 available from Rhodia;
^{3.}N-Hance 3196 polymer available from Aqualon;
^{4.}Polyox WSR 301 available from Dow Chemical;
^{5.}Iconal TDA-3 from BASF Corp.;
^{6.}Keltrol 1000, CP Kelco;
^{7.}Dissolvine NA 2x;
^{8.}Kathon CG, Rohm & Haas;
^{9.}Expancel 091 WE40 d24, Akzo Nobel;
^{10.}available from Quidesa, Mexico.

Prepare the compositions described above by conventional formulation and mixing techniques. First prepare a citric acid premix by adding citric acid into water at 1:3 ratio and a Polyox premix by adding Polyox WSR 301 and Keltrol 1000 to Trideceth-3 and Isosteareth-2. Then, add the following ingredients into the main mixing vessel with agitation: water, N-Hance 3196, Expancel, and Polyox premix. Then, add sodium trideceth sulfate, sodium lauroamphoacetate, sodium lauryl sulfate. Add sodium chloride, disodium EDTA, sodium benzoate, and Kathon CG. Adjust pH to 6.0 by adding citric acid premix. Then, add perfume and keep mixing until homogeneous. In a separate vessel, add Petrolatum and heat to 190 F. Add petrolatum into the main mixing vessel and keep mixing until homogeneous.

The following examples described in Table 6 and 7 are non-limiting examples of in-shower body lotion compositions of first personal care article of the present invention. All exemplified amounts are concentrations by weight of the total in-shower body lotion compositions, unless otherwise specified.

TABLE 6
In-shower body lotion composition of the Present Invention

| Ingredient | Example # | | | | | | |
|---|---|---|---|---|---|---|---|
| (by weight %) | 10 | 11 | 12 | 13 | 14 | 15 | 16 |
| Hydroxypropyl Starch | 3.5 | 4.0 | 3.5 | 3.5 | 4.0 | 4.0 | 3.5 |
| Phosphate^{1.} | | | | | | | |
| Emulsifying Wax NF^{2.} | 2.75 | 3.0 | 2.75 | — | 3.0 | 3.0 | 1 |
| Tween 60^{3.} | — | — | — | 0.5 | — | — | — |
| Cetyl Alcohol | — | — | — | 0.4 | — | — | — |
| Stearyl Alcohol | — | — | — | 0.4 | — | — | — |
| Fragrance | 1.0 | 1.0 | 1.0 | 1.0 | 1.0 | 1.0 | 1.0 |
| Preservatives | 0.8 | 0.8 | 0.8 | 0.8 | 0.8 | 0.8 | 0.8 |
| Water | Q.S. | Q.S. | Q.S. | Q.S. | Q.S. | Q.S. | Q.S. |
| Petrolatum^{4.} | 15 | 25 | 20 | 20 | 15 | | 35 |
| Mineral Oil^{5.} | — | — | 5 | — | — | — | — |
| Jojoba Oil^{6.} | — | 5 | — | — | — | — | — |
| Silicone Fluid^{7.} | — | — | — | — | 2 | — | — |
| Gelled Mineral Oil^{8.} | — | — | — | — | — | 15 | — |

^{1.}Structure XL available from National Starch;
^{2.}Polawax available from Croda;
^{3.}Polysorbate-60 available from ISP;
^{4.}Superwhite Protopet available from WITC;
^{5.}Hydrobrite 1000 PO White MO available from WITCO;
^{6.}Lipovol J available from Lipo;
^{7.}50 cstk available from Dow Corning;
^{8.}Versagel M750 available from Penreco.

The in-shower body lotion composition of Example 10-16 can be prepared by conventional formulation and mixing techniques. One such example is shown below, although a variety of orders of addition can be used to formulate useable products. Prepare the aqueous phase composition by first dispersing the hydroxypropyl starch phosphate in water. Add gel network phase (emulsifying wax or tween 80/cetyl alcohol/stearyl alcohol blend) and heat to 160° F. Place mixing vessel in a water bath to cool to under 100° F. Add fragrance. Premix all lipids at 160° F. Add to the aqueous phase (<80° F.) with increased agitation. (In the case of examples with multiple lipids, the lipids can be premixed or not, depending upon the desired outcome.) Add preservatives and agitate until product is smooth.

TABLE 7
In-shower body lotion composition of the Present Invention

| | Example# | | | |
|---|---|---|---|---|
| Ingredient (by weight %) | 17 | 18 | 19 | 20 |
| Sepigel 305^{1.} | — | 0.5 | — | — |
| Hydroxypropyl Starch Phosphate^{2.} | 3.5 | 2.5 | 3.5 | 3.5 |
| Emulsifying Wax NF^{3.} | 3.0 | 2.25 | 3.0 | 2.0- |
| Fragrance | 1.0 | 1.0 | 1.0 | 1.20 |
| Preservatives | 0.8 | 0.8 | 0.8 | 0.65 |
| Water | Q.S. | Q.S. | Q.S. | Q.S. |
| Petrolatum^{4.} | 20 | — | — | 25 |
| Mineral Oil^{5.} | — | — | 5 | — |
| G-2180 Petrolatum^{6.} | — | 25 | 20 | — |
| Dimethicone | — | — | — | 2 |
| Shea Butter | — | — | — | .01 |
| Interference Pigments^{7.} | — | — | — | 0.7 |

^{1.}available from Seppic;
^{2.}Structure XL available from National Starch;
^{3.}Polawax from Croda;
^{4.}Superwhite Protopet available from WITCO;
^{5.}Hydrobrite 1000 PO White MO available from WITCO;
^{6.}available from Crompton;
^{7.}KTZ Interval Gold lls10 available from Kobo Products, Inc.

The in-shower body lotion composition of Example 10-12 can be prepared by conventional formulation and mixing techniques. One such example is shown below, although a variety of orders of addition can be used to formulate useable products. First, prepare the aqueous phase composition by dispersing the hydroxypropyl starch phosphate in water. Add gel network phase (emulsifying wax or tween 80/cetyl alcohol/stearyl alcohol blend) and heat to 160° F. Place mixing vessel in a water bath to cool to under 100° F. Add fragrance. Add the lipid(s) (preheated to 160° F.) to the aqueous phase (<80° F.) with increased agitation. (In the case of examples with multiple lipids, the lipids can be premixed or not, depending upon the desired outcome.) Add preservatives and agitate until product is smooth.

Examples of the kit of the present invention is the combination of one of the personal care compositions from Tables 4-5, one of the in-shower body lotions from Table 6-7, and a shaving razor, such as a Venus® razor.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A shaving kit comprising:
(a) a first personal care article comprising:
1. a personal care composition a package containing; said personal care composition comprising:
a. a surfactant,
b. water, and
c. at least about 15%, by weight of personal care composition, of a hydrophobic moisturizing material;
(b) a second personal care article comprising:
1. an in-shower body lotion composition contained in a package; and
2. a set of instructions in association with said package, said set of instructions comprising instructions to dispense said in-shower body lotion composition from said package, contact skin surface with said in-shower body lotion composition during showering or bathing, rinse said skin surface, and dry said skin surface; and
(c) a shaving razor.

2. The shaving kit of claim 1, further comprising a plurality of replacement razor blades.

3. The shaving kit of claim 2, further comprising a storage device for storing said shaving razor and said plurality of said razor blades.

4. The shaving kit of claim 2, further comprising a dispenser for dispensing said plurality of razor blade cartridges.

5. The shaving kit of claim 1, further comprising one or more additional packages containing a personal care composition selected from the group consisting of a shampoo, conditioner, body lotion, skin care composition, deodorant, antiperspirant, after shave lotion and mixtures thereof.

6. The shaving kit of claim 5, wherein said additional packages are a size selected from the group consisting of full size, travel size, trial size and mixtures thereof.

7. The shaving kit of claim 1, further comprising a coupon, rebate, or advertisement.

8. The shaving kit of claim 7, wherein said coupon, rebate or advertisement is associated with a personal care products selected from the group consisting of a shampoo, conditioner, body lotion, skin care composition, deodorant, antiperspirant, after shave lotion, shaving razor, razor blade cartridges, and mixtures thereof.

9. The shaving kit of claim 1, further comprising a grooming device selected from the group consisting of a cleansing cloth, disposable cleansing cloth, a pumice stone, a brush, a comb, a mirror, tweezers and mixtures thereof.

10. The shaving kit of claim 1, wherein said hydrophobic moisturizing material is selected from the group consisting of petrolatum, mineral oil micro-crystalline waxes, paraffins, ozokerite, polyethylene, polybutene, polydecene and perhydrosqualene, dimethicones, cyclomethicones, alkyl siloxanes, polymethylsiloxanes and methylphenylpolysiloxanes, lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate castor oil, soy bean oil, sunflower seed oil, maleated soy bean oil, safflower oil, cotton seed oil, corn oil, walnut oil, peanut oil, olive oil, cod liver oil, almond oil, avocado oil, palm oil and sesame oil, and mixtures thereof.

11. The shaving kit of claim 1, wherein said personal care composition comprises at least about 20%, by weight of personal care composition, of a hydrophobic moisturizing material.

12. The shaving kit of claim 1, wherein said first personal care article comprises a set of instructions associated with said package comprising the step of using said personal care composition during shaving.

13. The shaving kit of claim 1, wherein said second personal care article further comprises a set of instructions associated with said package comprising the step of using said in-shower body lotion composition after shaving.

14. The shaving kit of claim 1, comprising a set of instructions associated with said kit comprising the following steps:
1. wetting a skin surface:
2. dispensing a personal care composition from a first personal care article;
3. contacting the skin surface with said personal care composition;
4. applying a shaving razor to said skin surface;
5. rinsing said skin surface of said personal care composition;
6. dispensing a in-shower body lotion composition from a second personal care article;
7. contacting said skin surface with said in-shower body lotion; and
8. rinsing said skin surface.

15. A method of shaving comprising the steps of:
1. wetting a skin surface:
2. dispensing a personal care composition from a first personal care article comprising a package containing said personal care composition;
3. contacting the skin surface with said personal care composition;
4. applying a shaving razor to said skin surface;
5. rinsing said skin surface of said personal care composition;
6. dispensing a in-shower body lotion composition from a second personal care article comprising a package containing an in-shower lotion composition;
7. contacting said skin surface with said in-shower body lotion; and
8. rinsing said skin surface.

16. The method of shaving of claim 15, wherein said method further comprises rubbing said personal care composition on the skin to form lather.

17. The method of shaving of claim 15, wherein said personal care composition is a visually distinct, multiphase personal case composition comprising a visually distinct cleansing phase and a visually distinct benefit phase which are in physical contact within said package.

18. A personal care article comprising:
1. an in-shower body lotion composition contained in a package; and
2. a set of instructions in association with said package, said set of instructions comprising instructions to dispense said in-shower body lotion composition from said package, contact skin surface with said in-shower body lotion composition during showering or bathing and after shaving, rinse said skin surface, and dry said skin surface.

19. The personal care article of claim 18, wherein said further comprise a coupon, rebate, or advertisement.

20. The personal care article of claim 19 wherein said coupon, rebate or advertisement is associated with a personal care products selected from the group consisting of a shampoo, conditioner, body lotion, skin care composition, deodorant, antiperspirant, after shave lotion, shaving razor, razor blade cartridge, and mixtures thereof.
